(19) United States
(12) Patent Application Publication
(10) Pub. No.: US 2010/0132707 A1
(43) Pub. Date: Jun. 3, 2010

(54) METHOD FOR CONTROLLING A TNI APPARATUS AND CORRESPONDING TNI APPARATUS

(76) Inventor: Ingo Muller, Dresden (DE)

Correspondence Address: LAW OFFICES OF CLEMENT CHENG, 17220 NEWHOPE STREET #127, FOUNTAIN VALLEY, CA, 92708, US

(21) Appl. No.: 12/298,056

(22) PCT Filed: Apr. 24, 2007
(86) PCT No.: PCT/DE2007/000727

§ 371 Date: Feb. 19, 2010

(30) Foreign Application Priority Data
Apr. 24, 2006 (DE) 10 2006 019 402.0

(51) Int. Cl. A61M 16/16; A61M 16/06; A61M 16/10

(52) U.S. Cl. 128/204.17; 128/207.18

(57) ABSTRACT

This invention relates to methods for controlling a TNI-apparatus (1). One method can measure the actual gas temperature at the outlet of the humidifier (19). One method can determine a set gas temperature (34) of the gas at the outlet of a humidifier (19) of the TNI-apparatus (1) in dependence on an ambient temperature (25) of the TNI-apparatus (1), or a set gas temperature (34) of the gas at the outlet of a humidifier (19) of the TNI-apparatus (1) in dependence on a gas flow (28) in the TNI-apparatus. One method can determine a set gas temperature (34) of the gas at the outlet of a humidifier (19) of the TNI-apparatus (1) in dependence on a comfort value (30) predetermined by a user, or adjust the heating power of a heating wire (26) in a nasal cannula (27) of a TNI-apparatus (1) in such a way that a gas exiting the nasal cannula (27) has approximately the same temperature, which the gas had when it exited a humidifier (19) of the TNI-apparatus (1). In addition, the invention relates to corresponding TNI-apparatus.

The invention relates to apparatus for the transnasal inspiration, which shall hereinafter be referred to as TNI-apparatus. Specifically, the invention relates to methods for controlling TNI-apparatus and to controlling TNI-apparatus.

The field of the invention specifically relates to TNI-apparatus according to the preambles of patent claims 16, 19, 21, 23, 24, 26 and 28.

TNI-apparatus are known, for example, from WO 02/062413 A2, in which they are referred to as anti-snoring apparatus. Such anti-snoring apparatus effect a splinting of the upper respiratory tract by administering air into the nose of a user through a conventional or modified oxygen cannula. Thus, the pressure in the respiratory tract is increased by some mbar above the ambient pressure.

The CPAP-therapy (continuous positive airway pressure) works similarly, whereby nose or face masks are used to administer the air at a pressure of around 5 mbar and at a maximum pressure of 30 mbar. As the masks are pressed against the face during the night, i.e. over a long period of time, by exerting a certain pressure, skin irritations may occur and, as a result, problems may arise in the acceptance by the patient.

Moreover, evaporators, specifically respiratory humidifiers, are known. In combination with the present invention, the evaporator known from WO 2006/012877 A1 can be used particularly advantageously.

In CPAP-apparatus, frequently radial blowers for conducting air are applied. Due to the smaller tube diameters and, as a result thereof, the higher pressures, side channel compressors are suited better for TNI-apparatus. Low-noise nasal cannulas, which are specifically suited for high gas, in particular airflows, are described in PCT/DE 2005/002335. These nasal cannulas additionally comprise a heating wire to avoid a condensation in the tubes of the nasal cannula.

It is the object of the invention to provide methods for TNI-apparatus and TNI-apparatus, which the users are willing to use, that is, which involve fewer problems as regards the acceptance by the users or patients.

This object is achieved with the teaching of the independent claims.

Preferred embodiments of the invention are defined in the dependent claims.

One advantage in measuring the gas temperature at the outlet of the humidifier is that it can be decided on the basis of the gas temperature as to whether the administered air is agreeable to the user. In a surprisingly advantageous manner, this gas temperature can be used as actual value in a control circuit for controlling the humidifier heating.

Moreover, it is surprisingly advantageous to adjust the heating power of the humidifier and/or the tube heating power in dependence on the ambient temperature so as to compensate for heat losses to the ambiance and prevent condensation in the nasal cannula.

It has been found in test series that a gas temperature above the ambient temperature by approximately 10 K is agreeable to a plurality of test subjects.

In addition, it is surprisingly advantageous to take into account the airflow in the control of the humidifier heating power and/or the tube heating power so as to prevent a temperature of the administered gas which is unpleasant to or dangerous for the user, a condensation in the nasal cannula or a destruction of the nasal cannula.

Furthermore, it is advantageous to provide the user with certain adjusting possibilities on the TNI-apparatus. The indirect adjustment of the gas temperature on the basis of a comfort value surprisingly provides for the possibility, if ambient parameters are changed, in particular the ambient temperature, and other settings, like the gas flow, to adjust the gas temperature in such a way that it will be agreeable to the user along with the new ambient parameters or settings, without a change to the comfort value.

The same parameters used to control the heating power of the humidifier can, in a surprisingly advantageous manner, also be used to control the tube heating power.

It is advantageous to adjust the tube heating power in such a way that it just about compensates the heat loss of the gas to be administered in the nasal cannula. In this case, the user is provided with a gas as humid as possible, which is agreeable to him and will not cause a condensation in the nasal cannula. In a wide range of the characteristic curve a slope of the tube heating power of −2% of the maximum heating power at a gas flow of 10 l/min per a gas flow difference of 1 l/min results in that the gas temperature at the outlets of the nasal cannula produces an agreeable sensation.

Switching off or at least reducing the tube heating power to below a gas flow of 10 l per minute advantageously prevents the heating wire from melting into the material (e.g. TPE or silicone) of the tubes of the nasal cannula or into the insulation of the heating wire, if a tube is kinked and the airflow for cooling the heating wire at the kink is no longer sufficient.

Below, a preferred embodiment of the invention will be explained in more detail by means of the accompanying drawings. In the drawings:

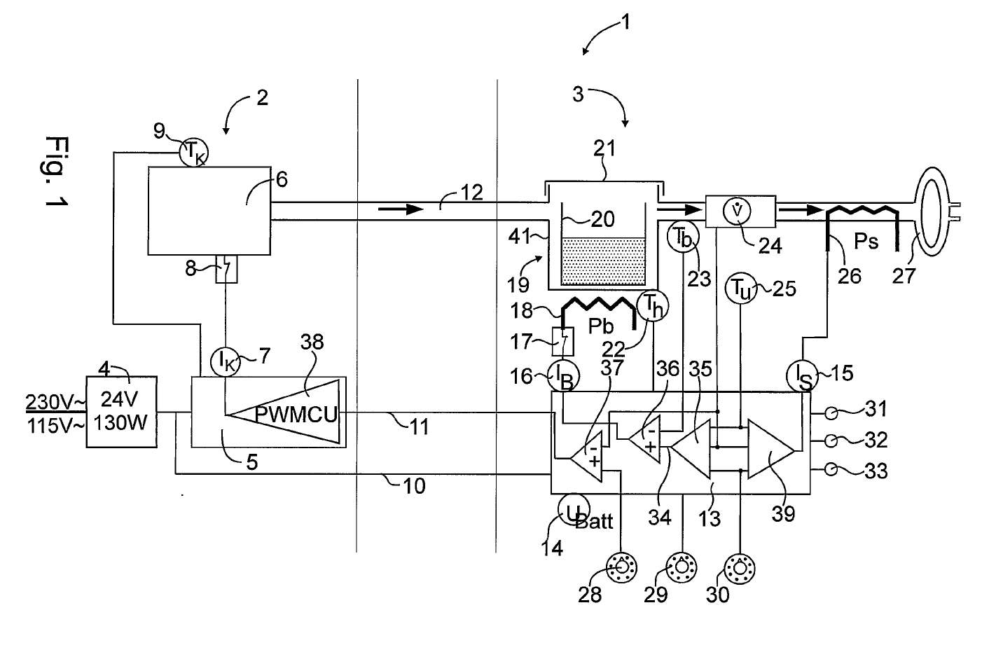

FIG. 1 shows a simplified circuit diagram of a TNI-apparatus according to the invention;

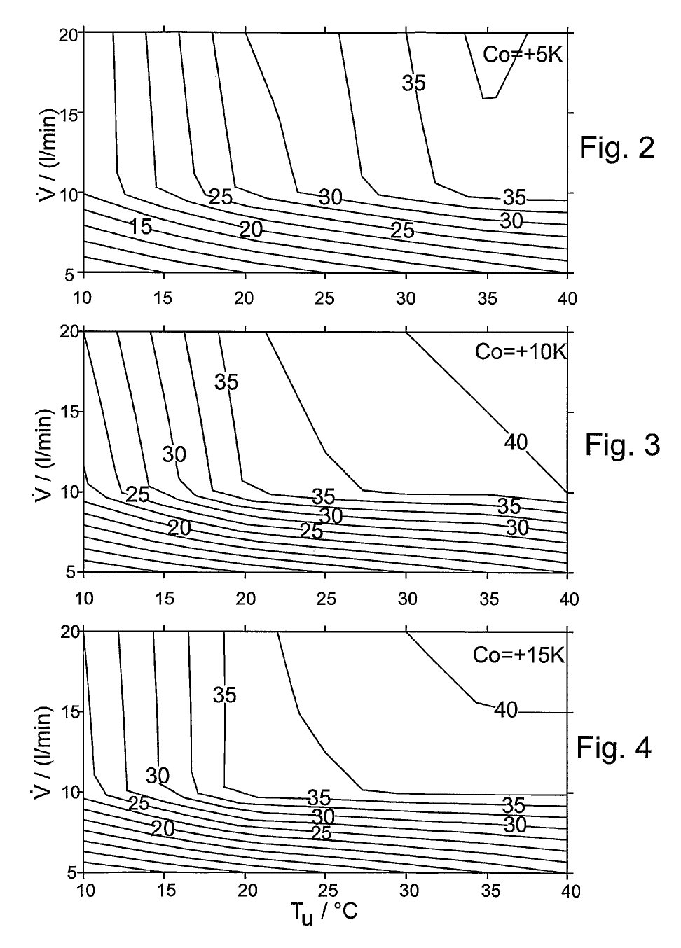

FIG. 2 shows the set gas temperature at a comfort value of 5K;

FIG. 3 shows the set gas temperature at a comfort value of 10K;

FIG. 4 shows the set gas temperature at a comfort value of 15K;

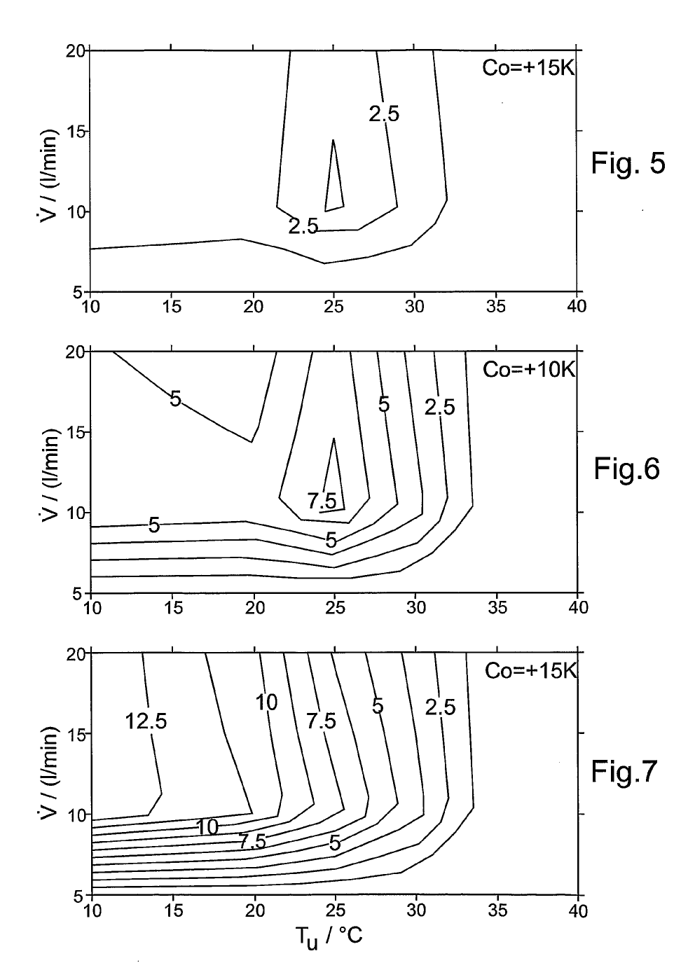

FIG. 5 shows the tube heating power at a comfort value of 5K;

FIG. 6 shows the tube heating power at a comfort value of 10K;

FIG. 7 shows the tube heating power at a comfort value of 15K;

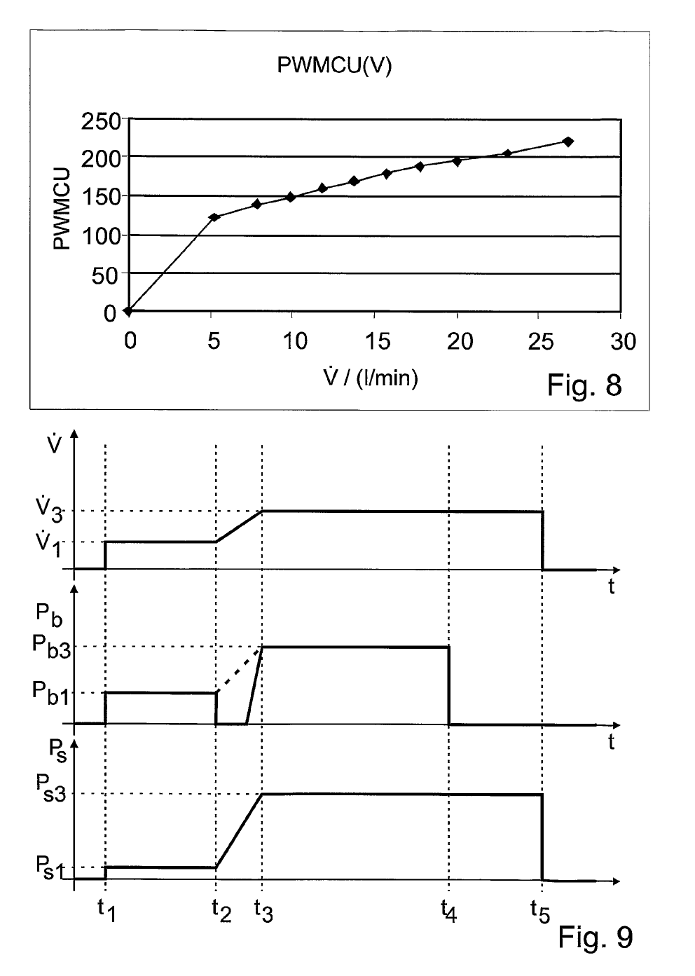

FIG. 8 shows a compressor function; and

FIG. 9 shows an on-off process.

FIG. 1 shows a simplified circuit diagram of a TNI-apparatus 1 according to the invention. In this document, a TNI-apparatus is an apparatus suited for transnasal inspiration. The TNI-apparatus 1 is comprised of a compressor unit 2 and a humidifier unit 3, which are connected to each other by a supply voltage connection 10, a data connection 11 and an airway 12. A serial interface is used as data connection 11.

The humidifier unit 3 comprises a humidifier 19, a gas temperature sensor 23, a volume flow sensor 24, a nasal cannula 27, an ambient temperature sensor 25 and a humidifier electronics 13. At present, an exactness of the gas temperature sensor 23 of ±1K is considered sufficient. The volume flow sensor AWM92100 24 of the company Honeywell as used herein works according to the by-pass principle and, therefore, has no dead spaces, so as to ensure a reliable disinfection.

The humidifier 19 may be constructed as the humidifier described in WO 2006/012877 A1. The humidifier 19 in FIG. 1 is depicted merely schematically and comprises a reservoir 20 for receiving evaporating liquid, in particular water, a lid 21 sealing the humidifier housing 41 in a pressure-tight manner, a humidifier heating 18 provided on the outside of the humidifier housing 41, a humidifier temperature sensor 22 provided in close thermal contact with the humidifier heating 18 and, for safety reasons, a temperature switch 17, which is likewise provided in close thermal contact with the humidifier heating 18.

The nasal cannula 27 may be constructed as the one described in PCT/DE 2005/002335. Specifically, a heating wire 26 is passed through the tubes of the nasal cannula, by means of which heat losses through the tubes to the ambiance can be compensated.

In the humidifier electronics a Hitachi H8S/HD2328 microcontroller is used. This microcontroller includes an integrated analog-digital converter, to which the analog signals of the volume flow sensor 24, of the humidifier temperature sensor 22, the voltage of a battery and the amplified voltage drops at series resistors to the heating wire 26 and the humidifier heating 18 are supplied. The series resistors permit a measurement of the currents through the heating wire 26 and the humidifier heating 18, respectively, and a detection of defects. In another embodiment, also digital sensors can be used.

The battery supplies a watch module and static memory chips (SRAM) with current when the TNI-apparatus is switched off. The battery itself is not shown in FIG. 1. Merely the battery voltage sensor 14 is illustrated.

Connected to the microcontroller are three rotary pulse generators without limit stop, namely a gas flow generator 28, a start delay generator 29 and a comfort value generator 30. In addition, three push-buttons 31, 32 and 33 are provided as operating elements, as well as a non-illustrated two-line LCD display with a width of 20 letters. By means of the gas flow generator 28 a gas flow between 10 l per minute and 20 l per minute can be adjusted. The comfort value generator 30 serves to adjust a comfort value, which will be explained in connection with FIGS. 2 to 7. The start delay generator 29 serves to adjust the time as of which the flow is raised from zero to its set value.

Although control and regulation take place digitally and program-controlled, the essential control and regulating functions are illustrated as triangles in the box of the humidifier electronics 13. Of greatest significance for the invention is the set gas temperature function 35, which calculates the set gas temperature at the outlet of the humidifier 19 from the gas flow measured by the volume flow sensor 24, from the comfort value Co adjusted by the comfort value generator 30 and from the ambient temperature Tᵤ measured by the ambient temperature sensor 25. This will be entered into in more detail below in connection with FIGS. 2 to 4.

The gas temperature controller 36 is supplied with the set gas temperature T_{b} at the outlet of the humidifier 19 and the actual gas temperature measured by the gas temperature sensor 23. The gas temperature controller 36 controls the humidifier heating power P_{b} supplied to the humidifier heating 18 in such a way that the set gas temperature and the actual gas temperature best possibly coincide with each other. A digital temperature sensor is provided as gas temperature sensor 23, inter alia, because of the small susceptibility to faults caused by electromagnetic interferences. If the microcontroller applied comprises a sufficient number of analog inputs or if the inputs are multiplexed, also an analog gas temperature sensor may be employed as gas temperature sensor 23. The control of the heating power itself is accomplished with pulse width modulation (PWM) in an external module. For the sake of EMC-compatibility the switching frequency was reduced to a few hertz. By means of reservoir capacitors the switching edges are smoothed, so that a direct voltage with residual ripple is applied to the heating. The gas temperature controller 36 optimally has a PID (proportional, to integral, differential) characteristic. In other embodiments, however, also an integral and/or proportional controller may be used.

Another important aspect of the invention is the tube heater control 39, which controls the tube heating power supplied to the heating wire 26 in the nasal cannula 27. The tube heater control, too, is supplied with the gas flow measured by the volume flow sensor 24, the comfort value adjusted by the comfort value generator 30 and the ambient temperature measured by the ambient temperature sensor 25. The control characteristic of the tube heater control 39 will be explained in more detail below in connection with FIGS. 5 to 7. The tube heating power substantially serves to compensate a temperature loss of the gas to be administered as it flows to the nasal cannula. The control of the heating wire 26 is likewise accomplished with a PWM of a few hertz, wherein the switching edges are likewise smoothed. As the heating wire 26 in the nasal cannula 27 forms a loop, the emission of electromagnetic interferences is here particularly critical.

The humidifier electronics 13 can, moreover, comprise a compressor controller 37, to which the flow signal of the volume flow sensor 24 and the gas flow adjusted by the gas flow generator 28 are supplied. The output signal of the compressor controller 37 is supplied via the data connection 11 to the compressor electronics 5, in particular to a compressor function 38. An example of the compressor function 38 is shown in FIG. 8. The compressor function 38 linearizes the characteristic curve of the compressor 6, so that the humidifier electronics 13 can request via the data connection 11 a specific gas flow. The value PWMCU is proportional to the pulse duty factor by means of which the motor of the compressor 6 is controlled, wherein a value of 255 corresponds to a pulse width of 100% and, thus, to the maximum motor and compressor power.

In order to preclude any risk for the user, the temperature of the compressor is detected by a digital compressor temperature sensor 9 and the current through the motor by a motor current sensor 7. The motor current sensor 7 is formed of a low-impedance resistor, which is connected in series with the motor, as well as of an amplifier and a low-pass filter. The amplifier adapts the low voltage dropping at the resistor to an analog input of the microcontroller used in the compressor unit 2. As microcontroller in the compressor unit 2 the AT90S2313 or a successor is envisaged. Furthermore, the motor speed is determined by internal Hall sensors.

The switched-mode power supply 4 supplies both the compressor unit 2 and the humidifier unit 3 via the supply voltage connection 10 with a direct voltage of 24 V. The three-phase compressor motor Papst ECA27.25 is directly operated with 24 V by a corresponding inverter, which also performs a pulse width modulation. For the control electronics itself the supply voltage is once more reduced to 12 V and 5 V. Moreover, +3.3 V and −3.3 V are made available in the humidifier unit. According to the regulation EN 60601-1 all poles of the power supply are disconnected from the mains supply by the switch on the backside of the apparatus (all poles power disconnection).

In the humidifier electronics 13 compliance data about the use of the TNI-apparatus by the user may be stored and read out by a USB (Universal Serial Bus) interface of the humidifier electronics 13. The USB interface is galvanically insulated, so as to preclude computers not complying with EN 60601-1.

In the stand-by mode, stand-by appears on the display. All devices of the TNI-apparatus 1 using a considerable amount of energy are switched off. In the operating mode, the date and hour as well three icons for the gas flow, the comfort value and the start delay, respectively, are displayed on the display. Pressing the stand-by push-button 33 permits the switching from the stand-by mode to the operating mode and vice versa. If one of the three shaft encoders is operated, a bar appears in the upper line of the display, illustrating by means of its width the value adjusted, and a description of the operated shaft encoder appears in the lower line. This mode is exited again after some seconds without user interaction.

To program the apparatus, which includes at least the setting of the parameters date and hour, the TNI-apparatus is transferred into the programming mode by pushing the first push-button 31. By pushing the second push-button 32, the parameters are cyclically advanced. The displayed parameter flashes and is altered by rotating the gas flow generator 28. By pushing the first push-button 32, the parameters are stored and the programming mode is exited.

As was mentioned before, the set gas temperature at the outlet of the humidifier 19 is determined by means of the set gas temperature function 35 in dependence on the adjusted gas flow, the adjusted comfort value and the measured ambient temperature Tᵤ. The set gas temperature function depending on three parameters is illustrated in FIGS. 2 to 4. Moreover, it is similarly illustrated in FIGS. 5 to 7 how the tube heating power Pₛ is likewise adjusted in dependence on the adjusted gas flow, the adjusted comfort value and the measured ambient temperature. In FIGS. 2 to 7, the flow \dot{v} in l/min is plotted on the Y-axis and the ambient temperature Tᵤ in ° C. is plotted on the X-axis. In FIGS. 2 to 4, set gas temperature T_{b} isotherms are plotted, wherein the temperature difference between two adjacent curves is 2.5 K and the numbers in the diagram indicate the set gas temperature in ° C. In FIGS. 5 to 7, lines of equal tube heating power Pₛ are plotted, wherein the numbers indicate the tube heating power in W and a point is used as decimal separator. The spacing between two adjacent curves corresponds to a tube heating power difference of 1.25 W.

FIGS. 2 to 7 represent the result of extensive tests. It was the aim of these tests to adjust the humidity and temperature of the administered gas to be as agreeable to the user as possible.

It was found that a temperature of 10 K above the ambient temperature, at a relative air humidity of about 80%, was agreeable to the users. From this follows that the humidifier temperature must approximately equal to the temperature of the administered gas so as to obtain a relative humidity of 80%, and that the temperature of the administered gas in the tubes of the nasal cannula must not drop below the humidifier temperature to a great extent so as to avoid condensation. In fact, it follows from an exact analysis of the test data that the humidifier temperature of the used humidifier known from WO 2006/012877 A1 has to be a few K above the temperature of the administered gas to allow the administered gas to have a relative humidity of 80% when it exits the nasal cannula. Thus, the heating wire 26 is controlled in such a way that it nearly compensates heat losses to the ambiance of the nasal cannula 27.

To provide the users with another adjusting possibility, which is rather independent of ambient conditions, in particular of the ambient temperature, but leads to the same well-being, the comfort value Co was introduced. It indicates the temperature difference between the ambient temperature and the temperature of the administered gas. As was explained above, a temperature difference of 10 K is, as a rule, agreeable. This comfort value Co was chosen for FIGS. 3 and 6. In FIGS. 2 and 5, a comfort value of 5 K was adjusted. In FIGS. 4 and 7, a comfort value of 15 K was adjusted. In the presently contemplated TNI-apparatus the comfort value is not calibrated in K, but a bar of medium length in the display rather corresponds to a temperature difference of 10 K. A longer or shorter bar stands qualitatively for a greater or smaller temperature difference.

It is assumed that, in use, the gas flow \dot{v} is above 10 l/min. Should the gas flow drop below 10 l/min, it is likely that a tube of the nasal cannula is kinked. To prevent the kink from locally overheating and, thus, the heating wire 26 from melting into the tube material of the nasal cannula, both the set gas temperature and the tube heating power are reduced below 10 l/min. As is illustrated in FIGS. 5 to 7, this can take place approximately linearly, so that with a flow of 5 l/min or below the tube heating power is reduced to 0. The drop of the humidifier heating power is steeper because the humidifier heating power is switched off when the set gas temperature falls below the ambient temperature. In FIGS. 2 to 4 this is the case below approximately 8 l/min.

In the following, the operating range above a gas flow of 10 l/min will be discussed. According to approval provisions, the gas temperature at the outlet of the nasal cannula should not be above 41° C. Therefore, one can see in FIGS. 2 to 4 that at ambient temperatures Tᵤ of above (41° C. comfort value), that is 36° C. in FIG. 2, 31° C. in FIG. 3 and 26° C. in FIG. 4, the spacings between the set gas temperature isotherms become greater, so that the set gas temperature is gradually transferred into a saturation. In none of the figures a curve for 42.5° C. is shown, so that the set gas temperature is, in fact, limited to 41° C. Particularly in FIGS. 2 and 4, the set gas temperature isotherms above 10 l/min and at temperatures of below 30° C. and 20° C., respectively, extend approximately parallel to the Y-axis, so that here the dependence of the set gas temperature on the gas flow is small. In FIG. 3, the set gas temperature isotherms are inclined slightly more strongly, so that at the same ambient temperature the set gas temperature increases with the gas flow. Below an ambient temperature of 20° C. and above a flow of 11 l/min the increase of the set gas temperature is approximately 0.2 K/(l/min). In the transition range toward to the saturation of the set gas temperature between 20° C. and 30° C. the increase of the set gas temperature is approximately 0.1 K/(l/min) due to the greater spacing between the set gas temperature isotherms.

In FIGS. 5 to 7 one sees at gas flows above 10 l/min that the tube heating power is reduced as the ambient temperature Tᵤ increases because less additional heating is required due to the small temperature difference from the ambiance. The maximum of the heating power in FIGS. 5 and 6 at an ambient temperature of 25° C. resulted from the measurements. An explanation for this can presently not be given.

At gas flows above 10 l/min, in FIGS. 5 and 6 above 25° C. and in FIG. 7 in the total ambient temperature range, the lines of equal tube heating power show a strong descending slope. Therefore, at the same ambient temperature, the heating power decreases by 0.05 to 0.2 W/(l/min) as the gas flow increases. As was mentioned above, the tube heating power does not compensate the heat loss to the ambiance completely. If more gas flows, the gas transports more thermal energy into the tube, so that the tube heating power can be adjusted downward. As was explained in connection with FIGS. 2 to 4, in particular FIG. 3, the set gas temperature is additionally raised as the gas flow increases, which, at the same temperature of the administered gas, is bound to lead to a stronger cooling of the gas in the tubes of the nasal cannula and, thus, to a lower tube heating power.

FIGS. 2 to 7 describe the behavior of the TNI-apparatus, in particular the behavior of the set gas temperature function and the tube heating in the operating mode. After switching it on by the user, a start-up program is run through, before the apparatus is transferred into the operating mode. After switching it off by the user, the TNI-apparatus is initially transferred into a switch-off mode, before it is finally switched off. This will be explained by means of FIG. 9 below.

The start-up program is executed between times t₁ and t₃. After switching the TNI-apparatus on by pushing the stand-by push-button 33 at time t₁, the user is meant to fall asleep first, before the TNI-apparatus is transferred to the operating mode at time t₃. The user shall not wake up during the transfer into the operating mode. Therefore, as was mentioned above, it is possible to input by means of the start delay generator 29 a start delay time (t₂−t₁) in the range of 0 to 60 min, in which the TNI-apparatus is substantially inactive. Specifically, no significant gas flow is adjusted until time t₂, so that the administered gas will not be particularly unpleasant to the user, even if the gas temperature and the humidity are not yet optimally adjusted.

However, the start delay time is used to preheat the liquid stock in the basin. However, the TNI-apparatus shown in FIG. 1 does not comprise a temperature sensor to directly measure the temperature of the liquid in the humidifier. The power P_{b1} can be calculated from the following formula (1):

$${P}_{b 1} = {C}_{{H}_{2} O} \frac{{T}_{bs} - {T}_{u}}{{t}_{2} - {t}_{1}} + W ( {T}_{bs} - {T}_{u} ) \qquad ( 1 )$$

C_{H}_{₂}_{O} thereby designates the heat capacity of the liquid stock and the humidifier 19, T_{bs} the set gas temperature at the outlet of the humidifier 19, Tᵤ the ambient temperature, t₂−t₁ the start delay time and W the thermal conductivity between the humidifier heating and the ambiance. The term

$${C}_{{H}_{2} O} \frac{{T}_{bs} - {T}_{u}}{{t}_{2} - {t}_{1}}$$

considers the power serving to heat the liquid, the term W(T_{bs}−Tᵤ) considers heat losses to the ambiance, which are of even more weight the longer the start delay time is. Thereby, it is assumed that the liquid stock has the temperature of the ambiance at time t₁. This need not be so if the liquid stock has just been refilled. Moreover, the heat capacity C_{H}_{₂}_{O} is actually dependent on the filling level, but is rather assumed to be constant. At short start delay times t₂−t₁ and cold ambient temperatures Tᵤ the power P_{b}, may become greater than the power P_{b3}.

From the thermal resistance between the humidifier heating 18 and the liquid stock in the basin 20, the heating power and the temperature measured by the humidifier temperature sensor 22 conclusions can be drawn to the liquid temperature T_{F} according to formula (2):

$${T}_{F} = {T}_{u} + \frac{{P}_{b 1}}{{W}_{b}} \qquad ( 2 )$$

W_{b} thereby designates the thermal conductivity between the humidifier heating 18 and the liquid stock. The thermal conductivity may be subject to great fluctuations and may be hard to reproduce. Nevertheless this method is, above all, favorable if or as soon as the liquid temperature is approximately correct, so that the heating power P_{b} can be reduced. The liquid temperature T_{F} can be used as actual value in a control loop.

Moreover, the conducted gas has approximately the liquid temperature T_{F} at the outlet of the humidifier. The greater the gas flow, the faster and more accurately can the liquid temperature be measured by the gas temperature sensor 23. The gas flow \dot{v} has, first of all, the purpose to avoid a condensation in the volume flow sensor 24 and amounts to between 1 and 5 l/min.

During preheating the liquid stock, overshoots can be provoked to high temperatures and used for destroying pathogens. The three above-explained methods for preheating the liquid stock can also be combined.

The tube heating power Pₛ₁ remains switched off during the start delay time, i.e. between t₁ and t₂. A condensation in the tubes of the nasal cannula is tolerated. In another embodiment, the tube heating power Pₛ₁ may be adjusted to a maximum of 5 W.

At time t₂ the TNI-apparatus changes over into a ramp mode, in which the gas flow \dot{v} and the tube heating power Pₛ are raised, for example linearly, to the operating values \dot{v}₃ and Pₛ₃ determined in FIGS. 2 to 7.

The set gas temperature function 35 and the gas temperature controller 36 determine the humidifier heating power during the ramp mode. This has the result that the humidifier heating power drops at first to 0 and increases rapidly as of a flow of approximately 8-9 l/min. More desirable would be a linear function, which is shown in dashed lines in FIG. 9, but it is not absolutely necessary because the time period without humidifier heating power is very time-limited by the ramp period described below.

At time t₃, the TNI-apparatus changes over into the operating mode. The ramp period t₃−t₂ can be adjusted in the range of 10 s to 600 s as parameter in the TNI-apparatus.

The switch-off mode between times t₄ and t₅ serves, above all, to dry the nasal cannula by blowing, to thereby prevent condensation after the humidifier heating 18 is switched off. The switch-off mode is started by pushing the stand-by push-button 33 at time t₄. If the stand-by push-button 33 is pushed during the start-up program, a changeover into the switch-off mode takes place as well. As is illustrated in FIG. 9, the humidifier heating is switched off immediately during the switch-off mode, while the gas flow and the tube heating power are kept constant during the switch-off mode. The TNI-apparatus can be switched off completely, thereby terminating the switch-off mode, if the temperature measured by the gas temperature sensor drops below a threshold, which can be calculated, for example, as an arithmetic means from the set gas temperature when the user switches off the TNI-apparatus, and from the ambient temperature. In addition or alternatively, a maximum time for the switching-off mode can be programmed as parameter for the TNI-apparatus. Although gas has generally been mentioned so far, in particular ambient air is conducted through and administered by the TNI-apparatus according to the invention.

Above, the invention was explained in more detail by means of preferred embodiments. A person skilled in the art will appreciate, however, that various alterations and modifications may be made without departing from the spirit of the invention. Therefore, the scope of protection will be defined by the accompanying claims and their equivalents.

### LIST OF REFERENCE NUMBERS

1 TNI-apparatus
2 compressor unit
3 humidifier unit
4 switched-mode power supply
5 compressor electronics
6 compressor
7 motor current sensor
8 compressor temperature switch
9 compressor temperature sensor
10 supply voltage connection
11 data connection
12 airway
13 humidifier electronics
14 battery voltage sensor
15 tube current sensor
16 heating current sensor
17 humidifier temperature sensor
18 humidifier heating
19 humidifier
20 basin
21 lid
22 humidifier temperature sensor
23 gas temperature sensor
24 volume flow sensor
25 ambient temperature sensor
26 heating wire
27 nasal cannula
28 gas flow generator
29 start delay generator
30 comfort value generator
31, 32, 33 push-button
34 set gas temperature
35 set gas temperature function
36 gas temperature controller
37 compressor controller
38 compressor function
39 tube heater control
41 humidifier housing

1-28. (canceled)

29. A method for controlling a TNI-apparatus, comprising the steps of: determining a set gas temperature of the gas at the outlet of a humidifier of the TNI-apparatus in dependence on an ambient temperature of the TNI-apparatus.

30. The method of claim 29, wherein the set gas temperature is approximately 10 K above the ambient temperature and the set gas temperature is limited to a maximum of 41° C.

31. The method of claim 29, wherein the set gas temperature is determined in dependence on a gas flow in the TNI-apparatus.

32. The method of claim 31, further comprising the step of determining a set gas temperature of the gas at the outlet of a humidifier of the TNI-apparatus in dependence on a gas flow in the TNI-apparatus.

33. The method of claim 32, wherein the set gas temperature slightly increases as the gas flow increases.

34. The method of claim 32, wherein the set gas temperature is determined in dependence on a comfort value predetermined by a user.

35. The method of claim 31, wherein the comfort value indicates by how many Kelvin the set gas temperature is above the ambient temperature, wherein the set gas temperature is limited to a maximum of 41° C.

36. The method of claim 35, further comprising the steps of: adjusting the heating power of a heating wire in a nasal cannula of a TNI-apparatus in such a way that a gas exiting the nasal cannula has approximately the same temperature which the gas had when it exited a humidifier of the TNI-apparatus.

37. The method of claim 36, wherein the heating power of the heating wire decreases as the gas flow increases, wherein the slope is, a negative percentage of the maximum heating power per a gas flow difference of 1 l/min at a gas flow of 10 l/min, a predetermined ambient temperature and a predetermined comfort value.

38. The method of claim 36, wherein the beating power of the heating wire is switched off below a gas flow of 8 l/min.

39. The TNI-apparatus comprising a humidifier unit, the humidifier unit comprising: a humidifier comprising an inlet and an outlet for gas; a heating for the humidifier; a temperature controller for controlling the heating power supplied to the heating; an ambient temperature sensor for measuring an ambient temperature of the TNI-apparatus.

40. The TNI-apparatus of claim 39, wherein the temperature controller is electrically connected to the temperature sensor and the temperature controller adjusts the heating power in dependence on the signal provided by the ambient temperature sensor.

41. The TNI-apparatus of claim 40, wherein the TNI-apparatus further comprises a gas flow generator for adjusting the gas flow, wherein the temperature controller is electrically connected to the gas flow generator and the temperature controller changes the set gas temperature in dependence on the adjusted gas flow.

42. The TNI-apparatus of claim 39, wherein the wherein the temperature controller is electrically connected to the gas flow generator and the temperature controller adjusts the heating power in dependence on the adjusted gas flow, wherein the heating power increases, in proportion to gas flow.

43. The TNI-apparatus of claim 39, wherein the TNI-apparatus further comprises a comfort value generator for adjusting comfort value, wherein the temperature controller is electrically connected to the comfort value generator and the temperature controller changes the set gas temperature in dependence on the adjusted comfort value, wherein an increase of the comfort value increases the set gas temperature.

44. The TNI-apparatus of claim 43, wherein the temperature controller is electrically connected to the comfort value generator and the temperature controller adjusts the heating power in dependence on the comfort value, wherein the heating power is proportional to the comfort value.

45. The TNI-apparatus of claim 39, further comprising an ambient temperature sensor measuring an ambient temperature of the TNI-apparatus, wherein the tube heater control adjusts the electrical power in dependence on the ambient temperature.

46. The TNI-apparatus of claim 39, wherein the TNI-apparatus further comprises a comfort value generator for adjusting a comfort value, wherein the tube heater control is electrically connected to the comfort value, generator and the tube heater control adjusts electrical power in dependence on the comfort value, wherein a high comfort value results in a high heating power.

47. The TNI-apparatus of claim 39, wherein the tube heater control is electrically connected to the comfort value generator and the tube heater control adjusts the electrical power in dependence on the comfort value, wherein the electrical power is the higher, the higher the comfort value is.

48. The TNI-apparatus of claim 39, wherein the TNI-apparatus further comprises a gas flow generator for adjusting the gas flow, wherein the tube heater control is electrically connected to the gas flow generator and the tube heater control changes the electrical power in dependence on the adjusted gas flow.

49. A TNI-apparatus comprising: a nasal cannula, through which a heating wire extends; a tube heater control, which is electrically connected to the heating wire and supplies electrical power to the heating wire; a gas flow generator for adjusting the gas flow, wherein the tube heater control is electrically connected to the gas flow generator and the tube heater control adjusts the electrical power in dependence on the adjusted gas flow, wherein if the electrical power is directly proportional to comfort value.
